(19) United States
(12) Patent Application Publication
(10) Pub. No.: US 2003/0124503 A1
(43) Pub. Date: Jul. 3, 2003

(54) Pyruvate cardioplegia solutions for administration to the heart during cardiopulmonary surgery and methods of use thereof

(76) Inventors: Albert H. Olivencia-Yurvati, Southlake, TX (US); Robert T. Mallet, Weatherford, TX (US)

Correspondence Address: BAKER BOTTS L.L.P., 2001 ROSS AVENUE, SUITE 600, DALLAS, TX, 75201-2980, US

(21) Appl. No.: 10/032,728

(22) Filed: Dec. 28, 2001

(51) Int. Cl. A01N 1/02; A61K 38/28; A61K 31/70; A61K 31/19

(52) U.S. Cl. 435/1.1; 514/3; 514/23; 514/557

(57) ABSTRACT

The invention includes a novel cardioplegia solution in which pyruvate serves as the primary energy source. The solution may be used in any surgery where the heart must be arrested, but it is particularly useful in cardiopulmonary bypass surgery. Because the solution relies primarily upon pyruvate to protect the heart from damage during and immediately after arrest, other additives are not as necessary as with current cardioplegia solutions or are not necessary at all. The invention also includes a method for making the cardioplegia solution and a process of performing cardiopulmonary bypass surgery using the novel cardioplegia solution.

### BACKGROUND OF THE INVENTION

This invention concerns a novel pyruvate-containing cardioplegia solution and its use for arresting and preserving the heart during heart surgery. To arrest the heart, the solution is introduced into the heart's coronary blood vessels. Chemical components of the solution protect the heart from injury and preserve heart muscle tissue throughout the period of cardiac arrest, allowing for improved post-surgical recovery of the heart's contractile performance as compared to lactate or glucose-based solutions currently used in the art.

Heart surgeries, such as coronary artery bypass grafting, valve replacement and repair of structural defects of the heart, are delicate procedures demanding a high level of surgical precision. It is typically necessary to temporarily stop the heart beat during these procedures so that the organ remains motionless, thereby facilitating the surgeon's work. The heart beat is arrested by introducing into the heart's coronary blood vessels special aqueous solutions, termed cardioplegia solutions, which contain chemicals that interrupt the physiological processes that cause the heart to beat.

A mechanical pump assumes the heart's role of supplying blood to the rest of the body while the heart is arrested. However, the heart's own coronary blood flow is interrupted during cardiac arrest and is not restored by the mechanical pump; the heart becomes ischemic. Coronary blood flow is critical because it supplies the heart with the fuels and oxygen it needs to generate ATP, the heart's main source of chemical energy. ATP supplies energy for numerous cellular processes that enable the heart to pump blood and which sustain the cells of the heart muscle. Cardiac arrest minimizes the heart's energy demands by interrupting the major energy-consuming process, that of pumping blood, but some energy is still required to support other cellular functions. Consequently the heart's energy reserves are depleted, albeit slowly, during cardiac arrest, due to the lack of coronary blood flow.

The gradual expenditure of energy reserves during cardiac arrest threatens to injure the heart's muscle tissue. Attempts have been made to minimize this energy depletion by adding energy-yielding fuels to cardioplegia solutions. Thus, cardioplegia solutions have utilized glucose, lactic acid and amino acids to support energy production in the arrested heart.

Glucose and lactate are readily metabolized by the normal, healthy heart muscle, ie. that with normal coronary blood supply. However, these compounds are ineffective fuels when coronary blood flow and delivery of oxygen is interrupted. The lack of oxygen causes massive accumulation of the metabolic cofactor NADH. As NADH accumulates within the heart muscle, it restrains metabolic activities of the enzymes glyceraldehyde 3-phosphate dehydrogenase and lactate dehydrogenase, which are required to consume glucose and lactate, respectively. See Kobayashi and Neely, 1979. Eventually energy production from glucose and lactate ceases. Moreover, NADH is harmful to the arrested heart because it serves as a precursor of harmful oxygen free radical compounds which injure the heart muscle cells. See Mohazzab-H et al. 1997 and Vandeplassche et al., 1989.

Amino acids, particularly glutamate and aspartate, also have been tested as components of cardioplegia. See U.S. Pat. Nos. 4,988,515 to Buckberg and 5,290,766 to Choong. It has been proposed that these compounds could bolster the heart's energy-generating capacity by increasing activity of the Krebs cycle, the main energy-producing metabolic pathway in the heart cells. However, this pathway cannot generate energy when coronary blood flow and oxygen supply are interrupted, as during cardiac arrest.

Aside from energy depletion, oxygen-centered free radical compounds are another potential cause of injury to the heart muscle during and following cardiac arrest. See Bolli and Marban, 1999; Bolli, 1991. During cardiac arrest, changes in the metabolic composition of the heart muscle, including accumulations of calcium ions and the metabolic cofactor NADH, set the stage for formation of massive amounts of free radicals when oxygenated blood is reintroduced into the coronary blood vessels after grafting is complete. Free radical compounds readily react with the normal constituents of cells, leading to chemical modifications of numerous proteins and membrane phospholipids that adversely affect the function of these biomolecules. By these mechanisms, free radicals injure or even kill heart muscle cells, leading to loss of functioning heart tissue and myocardial infarction. Cells are equipped with antioxidant molecules that defend the cells from free radical attack, but these endogenous antioxidant reserves are limited and are inadequate to protect the heart from the enormous free radical assault triggered upon resumption of coronary blood flow. Conceivably, addition of antioxidant chemicals to cardioplegia solutions could help protect the heart by neutralizing harmful free radicals as they are generated. Neither glucose, lactate nor amino acids are antioxidants.

The addition of pyruvate to cardioplegia solutions overcomes the disadvantages of other fuels because of its unique effects on the energy metabolism and antioxidant biochemistry of the heart muscle cells. Unlike glucose and lactate, pyruvate does not generate NADH but instead lowers NADH concentration within the cell's cytoplasm, enabling glucose metabolism to continue during cardiac arrest or following restoration of coronary blood flow (Bünger et al., 1989). Pyruvate produces a substantially higher energy state in the heart muscle than do other fuels (Bünger et al., 1989; Tejero-Taldo et al., 1998; Mallet and Sun, 1999), thereby providing more chemical energy to be used by the heart to pump blood. These metabolic actions enable the pyruvate-treated heart muscle to recover its pumping performance more rapidly following surgery than heart muscle treated with other fuels (Bünger et al., 1989; Mallet, 2000).

In addition to serving as an excellent fuel for energy production, pyruvate is a powerful antioxidant in the heart. Pyruvate's unusual α-keto carboxylic acid chemical structure enables it to directly react with and neutralize harmful oxygen free radical compounds (Constantopoulos and Barranger, 1984). In addition, metabolic conversion of pyruvate to citrate (Mallet and Sun, 1999; Tejero-Taldo et al., 1999) indirectly increases the amount of glutathione, the main antioxidant chemical in heart muscle cells. Glutathione is maintained by the metabolic cofactor NADPH. Citrate generated from pyruvate increases the amount of NADPH in the heart cells by increasing the metabolic activity of two processes that produce NADPH, namely, isocitrate dehydrogenase and the hexose monophosphate pathway. NADPH is used to restore glutathione consumed to neutralize free radicals. By this mechanism, pyruvate bolsters the heart muscle's natural defenses against free radicals.

Previous researchers have sometimes included pyruvate as an energy source in cardioplegia solutions, but they have failed to recognize its protective and other properties and have therefore additionally included other protective components. These additional components render the solution more costly than need be and the unnecessarily complex formulation provides greater chance for errors in preparation and increases the chance for adverse reaction to the cardioplegia solution. For instance in U.S. Pat. No. 4,988,515 to Buckberg et al., pyruvate is a potential energy source for the claimed cargioplegic solution, as are glucose, fructose and malate. Because Buckberg failed to recognize the favorable actions of pyruvate, other modifications of the solution were made to avoid damage to the heart. Primarily these modifications include limiting the calcium ion concentration of solution to between 50 and 300 μmol and providing a mechanism to maintain the metabolizable substrate concentration at 400-1000 mg % and osmolarity at an increased level of 400-500 mOsmol. Because Buckberg added these protective measures, he clearly failed to recognize the protective potential of pyruvate as the primary metabolite alone, resulting in an overly complicated cardioplegia solution.

In another patent, U.S. Pat. No. 6,153,647 to Mallet et al., the use of pyruvate to treat cardiac trauma is disclosed. However, the pyruvate is used to increase the inotropic effects of a β-adrenergic agonist which is co-administered, i.e., the desired effect is to increase the heart's contractions. Thus, the composition would not be used during surgery where it is undesirable to stimulate the heart's function due to the primary objective of arresting the heart. Similar effects using the same type of composition are described in isolated guinea-pig hearts in Tejero-Taldo, et al., 1998 and 1999. The applicability, if any, of these results to in situ guinea pig hearts of human patients is also not clear.

Similarly, in Mallet, 2000, the use of pyruvate to increase contractile properties of stunned heart tissue is discussed. Based upon this information, one would administer a pyruvate solution to a heart which one wishes to resume beating, but not to an arrested heart which is to remain quiescent for surgery. Four additional studies, Bünger and Mallet, 1994 and 1993, Mallet et al., 1990 and Bünger et al., 1989, address the actions of pyruvate in non-ischemic or in stunned hearts, but fail to address its uses in an arrested heart.

Additionally, these studies in isolated guinea pig heart are of uncertain relevance to human heart surgery. Although such studies are useful in ruling out immediate harmful effects and indicating that some positive effect may be possible, because of physiological differences between mammals, and especially between small mammals such as guinea pigs and large mammals such as humans, it is far from certain that any useful effect seen in a guinea pig heart will be duplicated in a human heart. Also the extent of any useful effect is likely to vary a great deal among species.

Furthermore, in these guinea pig studies, the objective was to increase post-ischemic function of guinea pig hearts, whereas the in the present invention the cardioplegia is applied during ischemia to increase cardiac function at that time so the heart can recover more completely after the ischemia. The correlation between an increase in post-ischemic cardiac function and an increase in cardiac function during ischemia is not to such a degree that predictions of one effect may be based upon experiments to study the other.

Finally, there are obvious differences between using an isolated heart and a heart still located in a living organism which is intended to continue to function after the test. These differences alone make it nearly impossible to predict whether useful results will be obtained in a human surgery without doing such experiments in a human. Thus, guinea pig studies primarily serve to rule out severe safety implications and to indicate whether further experimentation using human patients is warranted.

Others have addressed the use of solutions containing chemical derivatives of pyruvate in post-ischemic events. For example, in three patents to Brunengraber et al., U.S. Pat. Nos. 5,667,962, 5,876,916, and 5,968,727, a pyruvate solution is used to treat reperfusion injury following ischemia. However, these patents use a pyruvate thiolester, which is chemically and functionally different from unmodified pyruvate. Additionally, in the Brunengraber patents the pyruvate derivative is administed only after, not during an ischemic event.

The capacity of pyruvate to act as an energy source for and/or to prevent free-radical damage in organs, in some cases including the heart, which require additional energy (due to disease or because they have been removed for transplant) or are in ususual danger of free-radical damage is discussed in several other patents. These include U.S. Pat. Nos. 6,143,784 to Greenhaff et al., 5,480,909 and 5,294,641 to Stanko, 6,086,789 to Brunengraber et al., 5,066,578 and 5,075,210 to Wikman-Coffelt et al., and 4,663,166, 5,100,677, and 6,020,007 to Veech. These patents disclose administration of pyruvate in a number of manners, many of which are topical, oral or intravenous. None of the patents disclose or suggest use of pyruvate in a carioplegic solution.

Thus, a need exists for a cardioplegia solution for use in cardiopulmonary surgery in which pyruvate is the major energy source and acts to prevent damage to the heart muscle.

All references cited in the above Background of the Invention are incorporated by reference herein.

### SUMMARY OF THE INVENTION

The invention provides a novel cardioplegia solution and its use in cardiopulmonary bypass surgery. The cardioplegia solution comprises water with the following chemicals dissolved therein in the following concentrations:

| | |
|---|---|
| Pyruvate (mM) | 0.2-50 |
| NaCl (mM) | 0-250 |
| KCl (mM) | 0-250 |
| Glucose (mM) | 0-200 |
| Insulin (U/l) | 0-200 |
| CaCl₂ (mM) | 0-20 |
| Lidocaine (g/l) | 0-2. |

The pyruvate may be provided in the chemical form of a free acid, as a salt in which the metal cation is sodium, calcium, or potassium, or as a salt in which the cation is an organic compound, for example, creatine. The cardioplegia solution may include additives to prevent bacterial growth or pyruvate breakdown. It may also include amino acids and/or vitamins. Although one benefit of the solution is that it provides protection to the heart through use of pyruvate as the principle energy source and without the need for other agents, at times it may be desirable to obtain added protection by including a pharmacological agent in the cardioplegia solution. Preferred pharmacological agents are β-adrenergic receptor antagonists, Ca²⁺ channel antagonists, and antioxidants.

The invention also includes a method of preparing the cardioplegia solution described above. The method consists primarily of dissolving medical-grade electrolyte reagents in water then filtering the solution through a filter with a pore size between of between 0.05 and 1 μm. Filtration removes contaminants and sterilizes the solution.

Another aspect of the invention relates to a process for performing cardiopulmonary bypass surgery in a human. The surgical process follows that or a routine cardiopulmonary bypass, except that the cardioplegia of the invention is used rather than a currently known cardioplegia. In general, the surgery begins with preparation of a patient to allow surgical access to the heart. Next, the heart is arrested through application of the cardioplegia solution described above. After arrest, a bypass mechanism is provided to a partially or wholly obstructed artery. When arrest is no longer necessary, administration of the cardioplegia solution is ceased and a chemical or mechanical stimulus is used to induce the heart to resume beating. The surgical procedure may then be completed.

In most bypass operations, it will be preferable to dilute the cardioplegia solution with whole blood prior to administration. The ratio of blood volume to cardioplegia solution volume may be between 0.1:1 and 20:1. It is preferably between 1:1 and 10:1 and more preferably between 2:1 and 8:1.

Administration of the novel cardioplegia solution during bypass surgery has numerous beneficial effects. In general, it protects the heart from injury resulting from ischemia. Thus, the heart exhibits rapid and robust recovery of mechanical function following the provision of a mechanical or chemical stimulus to resume beating.

These beneficial effects result from a number of causes. For instance, the cardioplegia solution stabilizes the heart's energy reserves during the cardiopulmonary bypass surgery. Additionally, metabolism of the cardioplegia solution by the heart produces compounds which neutralize prooxidant compounds during and immediately after the period of arrest. Metabolism of the cardioplegia solution by the heart also maintains the heart's antioxidant components during cardiopulmonary bypass surgery.

Because of these beneficial effects, in many bypass procedures pharmacological inotropic support need not be administered following completion of the surgical procedure. Otherwise, it may be administered in reduced amounts or frequency as compared with patients in which a lactate-based cardioplegia is used.

The above summary provides a general outline of the invention. For a better understanding of the invention and its advantages, reference may be made to the following description of exemplary embodiments, taken in conjunction with the accompanying drawings.

### BRIEF DESCRIPTION OF THE DRAWINGS

FIG. 1 presents data comparing the effects of a pyruvate-fortified cardioplegia solution of the present invention with those of a standard, lactate-fortified cardioplegia solution on hemoglobin oxygen saturation in coronary sinus blood (sampled as venous blood draining from the heart muscle). Data was obtained from a cohort of patients undergoing bypass surgery. In the graphs data from patients treated with the standard cardioplegia solution is shown in the left graph as circles while data from patients treated with the pyruvate-fortified cardioplegia solution is shown in the right graph as squares. The filled symbols represent individual patients; the open symbols represent mean values. “Pre-CPB” indicates measurements taken prior to cardioplumonary bypass surgery, while “Post-CPB” indicates measurements taken after surgery. Hemoglobin oxygen saturation indicates the concentration of oxygen in the heart muscle (Tenney, 1974, incorporated by reference herein).

FIG. 2 presents data comparing the effects of a pyruvate-fortified cardioplegia solution with those of a standard lactate-based cardioplegia solution on release of proteins (CPK-MB in Panel A and Troponin-1 in Panel B) from injured heart muscle cells. Samples were taken from venous blood draining from the heart muscle. Data was obtained from a cohort of patients undergoing bypass surgery. In the graphs data from patients treated with the standard cardioplegia solution is shown in the left graph as circles while data from patients treated with the pyruvate-fortified cardioplegia solution is shown in the right graph as squares. The filled symbols represent individual patients; the open symbols represent mean values. “Pre-CPB” indicates measurements taken prior to cardioplumonary bypass surgery, while “Post-CPB” indicates measurements taken after surgery. Release of heart muscle proteins increased after cardiac arrest with the standard cardioplegia solution but did not increase after arrest with the pyruvate-fortified cardioplegia solution.

FIG. 3 presents data comparing the effects of a pyruvate-fortified cardioplegia solution with those of a standard lactate-based cardioplegia solution on post-surgical recovery of left ventricular contractile function following cardioplegia arrest. In the graphs data obtained using standard cardioplegia solution is shown as circles while data obtained using pyruvate-fortified cadrioplegic solution is shown as squares. Data was obtained from a cohort of patients undergoing bypass surgery.

FIG. 4 presents the number of patients requiring pharmacological treatments to stimulate the heart muscle following bypass with a pyruvate-fortified cardioplegia solution or a standard lactate-based cardioplegia solution. Data was obtained from a cohort of patients undergoing bypass surgery.

FIG. 5 presents data regarding the stability of a pyruvate-fortified cardioplegia solution (square symbols) and a standard lactate-based cardioplegia solution (round symbols). The solutions were prepared from standard electrolytes and fortified with lactate or pyruvate. Solutions were filtered (0.22 μm pore) to remove contaminants, then maintained at room temperature (c. 22° C.; shown by filled symbols) or refrigerated (c. 4° C.; shown by open symbols). Acetate was monitored as a degradation product of the carbohydrates. Lactate, pyruvate and acetate were measured by colorimetric assays at the indicated intervals following preparation of the solutions. Values are the mean of three measurements. RT=Room Temperature.

### DETAILED DESCRIPTION OF THE INVENTION

The present invention includes a pyruvate-fortified cardioplegia solution for protecting the heart during cardiopulmonary bypass surgical procedures and its use in such procedures. The solution contains pyruvate anion at a concentration between 0.2 and 50 millimolar, and is administered into the heart's coronary blood vessels via the aorta and coronary sinus. This novel solution is superior to existing cardioplegia solutions because it both preserves the heart's energy resources, enabling the heart to pump blood more effectively following surgery, and it bolsters the heart's antioxidant defenses, providing better protection from harmful free radicals generated in the heart during surgery. Compared to other cardioplegia solutions, the use of this invention improves recovery of cardiac function following surgery.

The present invention achieves these favorable effects primarily through the use of pyruvate as the primary energy source in the cardioplegia solution. Although the invention may be employed in any type of surgery requiring cardioplegia solution, it is preferably used during cardiopulmonary bypass operations. Although the patients are preferrably human patients, the invention may be employed in non-human animals, particularly in other large mammals.

The cardioplegia solution of the invention may contain other additives to prevent damage to the heart, but a main advantage of the invention is that it provides protection via the energy source, eliminating or reducing the need for any additives or other changes in formulation to prevent heart damage.

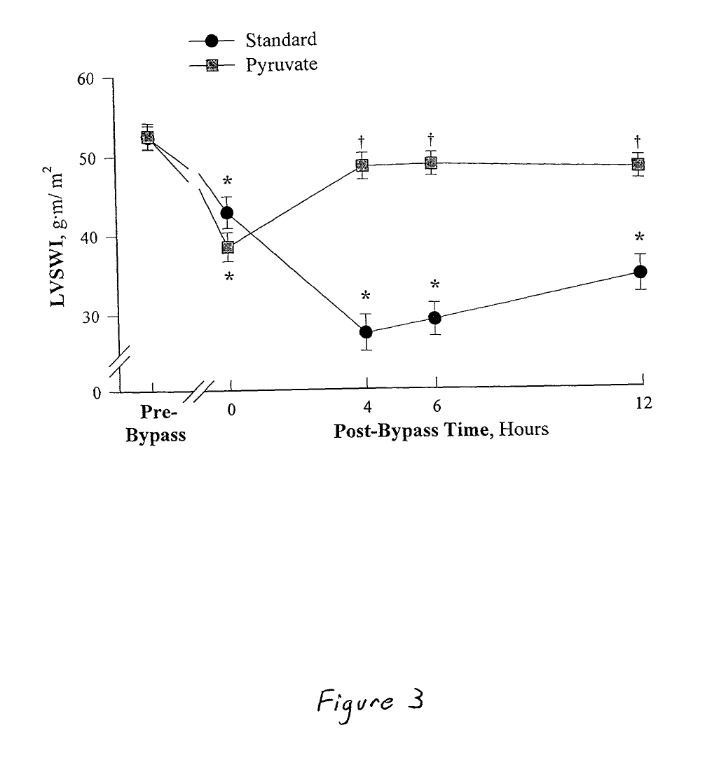

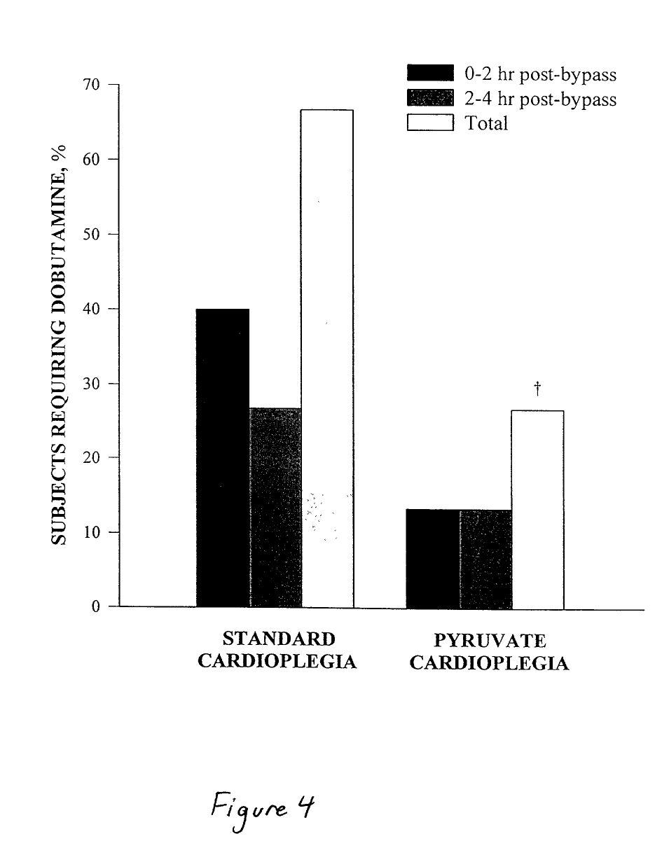

This invention will allow safer use of cardioplegia solution with improvement in early and presumably long-term functional recovery of the myocardium in patients. (See FIG. 3.) It should be noted that the invention has nearly eliminated the need for post-operative pharmacological inotropic support (See FIG. 4), presumably by preventing damage to the heart as a result of arrest and ischemia. Consequently, in tests this has shortened each patient's stay in the intensive care unit by an average of one full day, with a savings of approximately $3000 per patient. As healthcare costs continue to rise, even greater savings may later be achieved.

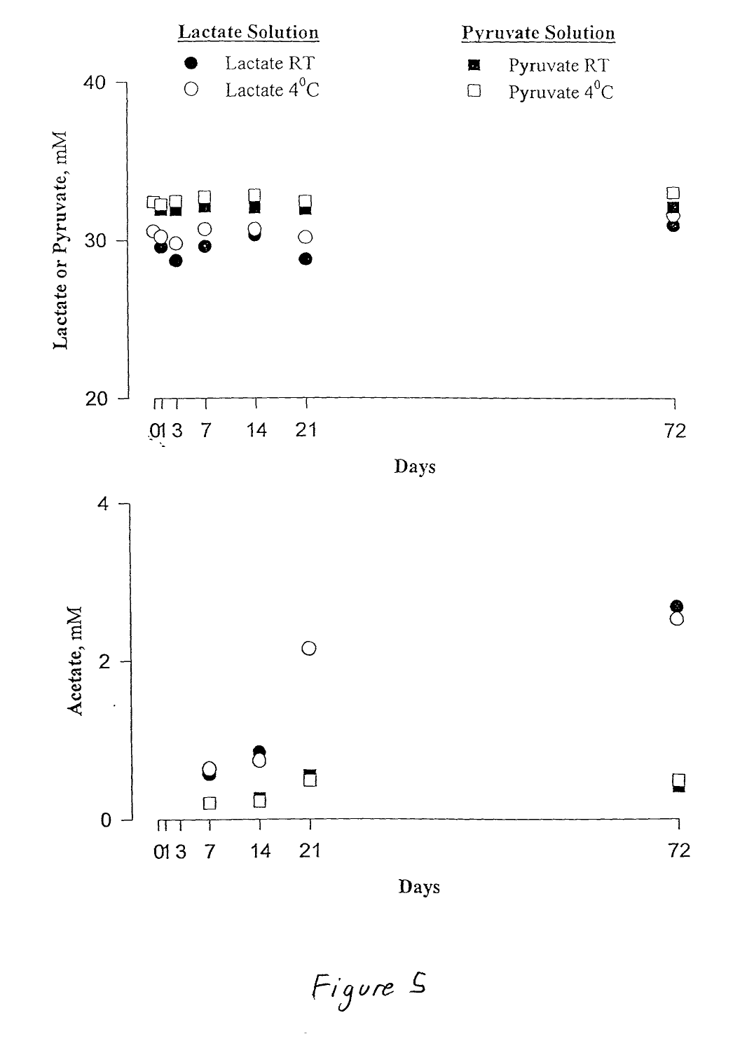

Furthermore, the cardioplegia solution of the present invention is no more difficult to make than currently used solutions. In fact, is is simpler than many solutions which attempt to achieve pyruvate's protective effects through careful control of solution concentrations or osmolarity, or by limitation or addition of ingredients. Additionally, it is at least as stable as a lactate-based solution (See FIG. 5), therefore it should have a shelf life similar to that of current solutions. Should a longer shelf-life be required, additivies known to the art and acceptable for cardioplegia solutions may be included. Thus, the added benefits of the cardioplegia solution of this invention may be obtained without added production costs and possibly with a reduction of costs. Because it is used in the same manner as other cardioplegia solutions, it adds no use costs.

The invention may be better understood through reference to the specific examples which follow. Although only preferred embodiments of the invention are specifically described above and in the examples, it will be appreciated that modifications and variations of the invention are possible without departing from the spirit and intended scope of the invention.

### EXAMPLES

### Example 1: Preparation of Lactate and Pyruvate-Based Cardioplegia Solutions

Cardioplegia Solutions were prepared using standard electrolyte reagents suitable for medical formulations. After preparation, the solutions were sterilized and contaminants removed by filtration through a 0.22 μm pore filter.

The components of the lactate-based solution are as follows:

| Ingredient | Quantity | |
|---|---|---|
| Lactated Ringers | 800 | ml (3140 mg Na lactate) |
| KCl (2 mEq/ml) | 108 | mEq (54 ml) |
| NaHCO₃ (1 mEq/ml) | 160 | mEq (160 ml) |
| Dextrose, 50% | 80 | ml |
| Regular Insulin | 80 | units (0.8 ml) |
| CaCl₂ injection, 10% | 800 | mg (8 ml) |
| Lidocaine 1% | 800 | mg (80 ml) |
| Total volume | 1182.8 | ml |

The components of the pyruvate-based solution are as follows:

| Ingredient | Quantity | |
|---|---|---|
| Pyruvated Ringers | 800 | ml (3080 mg Na pyruvate) |
| KCl (2 mEq/ml) | 108 | mEq (54 ml) |
| NaHCO₃ (1 mEq/ml) | 160 | mEq (160 ml) |
| Dextrose, 50% | 80 | ml |
| Regular Insulin | 80 | units (0.8 ml) |
| CaCl₂ injection, 10% | 800 | mg (8 ml) |
| Lidocaine 1% | 800 | mg (80 ml) |
| Total volume | 1182.8 | ml |

These concentrations of lactate and pyruvate are 35 mM in the Ringers solution, 23.7 mM in the cardioplegia solution.

### Example 2: Alternative Cardioplegia Solutions

Cardioplegia solutions were prepared on the day of surgery as described in Example 1. The control solution contained lactated ringers as the main energy source. In the study solution, pyruvated ringers was substituted for lactated ringers. Pyruvated ringers was prepared by dissolution of sodium pyruvate powder of highest commercially available purity (tissue culture grade, Sigma Chemical Co., St. Louis, Mo.) in sterile 0.9% NaCl. Both cardioplegia solutions contained 104 mM NaCl, 135 mM NaHCO₃, 91 mM KCl, 6 mM CaCl₂, 188 mM glucose, 68 U/l insulin, and 676 g/l lidocaine. Sodium lactate concentration in the control solution was 23.8 mM, and the study solution contained 10 mM sodium pyruvate After adjusting the pH to 7.8 at 37° C., particulate contaminants were removed by filtering the cardioplegia solutions through 0.5 μm cellulose nitrate filters.

Although the above solutions in Examples 1 and 2 represent specific preferred embodiments of the invention, it will be understood that alternative embodiments with different concentrations and/or ingredients remain within the scope of the invention.

Specifically, other embodiments may have the following concentrations of ingredients, wherein each of the concentration ranges is a preferred embodiment:

| Ingredient | Conc. 1 | Conc. 2 | Conc. 3 |
|---|---|---|---|
| Pyruvate (mM) | 0.2-50 | 1-30 | 30-45 |
| NaCl (mM) | 0-250 | 10-100 | 100-200 |
| NaHCO₃ | 0-250 | 10-100 | 100-200 |
| KCl (mM) | 0-250 | 10-100 | 100-200 |
| Glucose (mM) | 0-200 | 10-80 | 80-170 |
| Insulin (U/l) | 0-200 | 10-80 | 80-170 |
| CaCl₂ (mM) | 0-20 | 0.5-5 | 5-15 |
| Lidocaine (g/l) | 0-2 | 0.25-1 | 1-1.75 |

It will be understood to one skilled in the art that each concentration of each ingredient in the above list is a separate preferred embodiment. For instance, a solution may lie within the ranges of the third concentration for each ingredient except lidocaine, which may be present in the concentration of the second column, 0.25-1. The appropriate combination of ranges and specific concentrations within each range will vary depending upon a variety of factors. These may include whether and which other pharmaceutical compositions are administered to the patient before, during, or after the surgery or the age and condition of the patient. Each component other than pyruvate indicated above may be substituted by any chemical known to have similar effects on the heart. In a specific preferred embodiment, the concentration of CaCl₂ is between 0.5 and 20 mM.

Preferred additives to the cardioplegia solution of the invention include vitamins and amino acids, β-Adrenergic receptor antagonists, Ca²⁺ channel antagonists, and antioxidants. The pyruvate used to create the solution of the invention may be provided in the chemical form of a free acid, a salt in which the metal cation is sodium, calcium, or potassium, or as a salt in which the cation is an organic compound such as creatine.

### Example 3: Chemical Stability of the Cardioplegia Solution

Solutions prepared as described in Example 1 except with a pyruvate or lactate concentration of 30 mM were maintained at room temperature or at 4° C. for up to 72 days. Samples were tested at days 1, 3, 7, 14, 21 and 72. Concentration of acetate was measured. Acetate is a degradation product of carbohydrates and its presence indicates breakdown of pyruvate or lactate. Lactate and pyruvate concentrations were also measured as an indicator of carbohydrate stability. Acetate, lactate and pyruvate concentration were measured using calorimetric assays standard in the art. The results of these assays are presented in FIG. 5. The results indicate that the novel pyruvate-based cardioplegia solution is at least as stable as the standard lactate-based solution. Should greater carbohydrate stability or greater sterility be desired, pharmaceutically acceptable additives known to the art may be included in the solution. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “additive” refers to a diluent or other chemical with which the therapeutic is administered. The additive may be added to the composition at the time it is made or at a later time. Examples of suitable pharmaceutical additives are described in “Remington: The Science and Practice of Pharmacy”, formerly “Remington's Pharmaceutical Sciences” by E. W. Martin.

### Example 4: Human Studies

To demonstrate the utility and effectiveness of the invention for its intended application, the novel pyruvate-fortified cardioplegia solution was tested in patients undergoing open heart surgery for coronary bypass grafting. Cardioplegia solutions were prepared as described in Example 2. Patients were randomly assigned to one of two treatment groups: in one group, hearts were arrested with standard cardioplegia containing lactate and glucose as fuels, and in a second group cardiac arrest was induced with the novel pyruvate-enhanced cardioplegia. In all surgeries the cardioplegia was mixed with patient blood in a ratio of blood volume:cardioplegia volume of 4:1 prior to administration to the heart. The mixture ratio may be anywhere between 0.1:1 and 20:1 and is preferably between 1:1 and 10:1 and more preferably between 2:1 and 8:1.

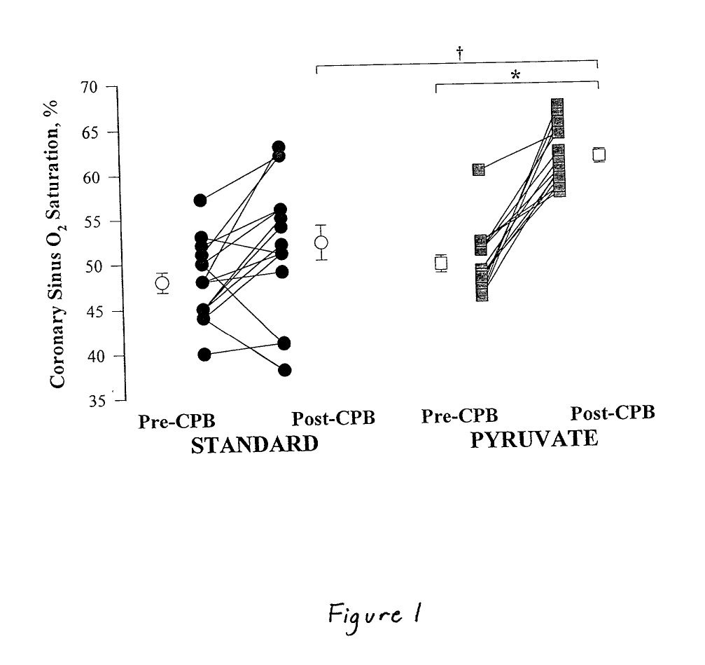

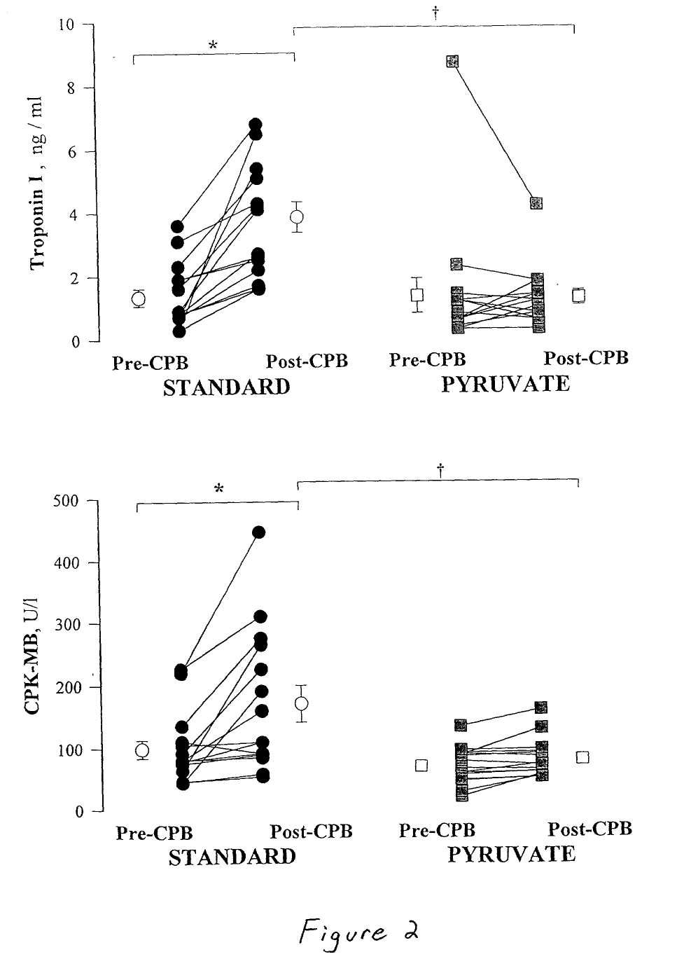

The protective effects of these solutions were compared by three criteria: coronary sinus hemoglobin oxygen saturation (FIG. 1), a measure of coronary blood flow and oxygen delivery to the heart muscle; coronary sinus troponin-I and creatine phosphokinase-MB isoform (CPK-MB) release (FIG. 2), which reflect the extent of injury to the heart muscle; and left ventricular stroke work index (FIG. 3), a measure of the heart's contractile performance. Coronary sinus oxygen saturation was similar in the two groups before bypass surgery, but after surgery oxygen saturation increased sharply in the pyruvate group but not in the standard cardioplegia group (FIG. 1). This indicates that use of the pyruvate-fortified cardioplegia solution improved the supply of oxygen to the heart muscle after surgery. Release of the cardiac muscle proteins troponin-I and CPK-MB increased in the standard group following surgery (FIG. 2), indicating that the heart muscle was injured during the period of cardiac arrest. Pyruvate cardioplegia prevented the post-arrest increases in troponin-I and CPK-MB release. Thus, use of the novel pyruvate-fortified cardioplegia prevented cellular injury while the coronary blood supply was interrupted during cardiac arrest.

Cellular protection by pyruvate during cardiac arrest was manifest by improved contractile performance of the heart during the post-arrest recovery period (FIG. 3). Cardiac function was similar in the two groups immediately after coronary blood supply was reinstituted and the heart weaned off of bypass, but function of the two groups differed remarkably by 4 hours post-bypass. Thus, left ventricular stroke work index declined between zero and four hours post-bypass in the standard cardioplegia group, but, in contrast, it increased considerably during the same period in the pyruvate cardioplegia group. Left ventricular function remained robust at least until 12 hours post-bypass in the pyruvate cardioplegia group, in contrast to the weak contractile function of hearts treated with standard cardioplegia. Thus, superior protection of the heart by pyruvate-fortified cardioplegia during cardiac arrest led to dramatically improved cardiac function post-arrest. This improvement in cardiac recovery obviated the use of dobutamine to pharmacologically stimulate cardiac function (FIG. 4). Thus, only 4 of the 15 pyruvate trials required any dobutamine to stimulate the heart post-bypass, but 10 of the 15 standard cardioplegia patients required pharmacological support. Dobutamine could potentially deplete the heart's energy reserves and thereby worsen injury to the heart, so the reduction in its use afforded by pyruvate is desirable.

It is important to note that in the method of this invention pyruvate is being given during the period of myocardial ischemia created by surgical arrest, as opposed to administration after the period of ischemia has ended (the so-called ‘reperfusion’ period). Ischemia and reperfusion are very different situations for the heart muscle. During ischemia, the coronary blood supply is interrupted, and, consequently, the heart muscle is deprived of its fuel and oxygen supplies. This is the situation the heart is subjected to during coronary bypass surgery. During reperfusion, the heart's blood supply has been restored, so the heart once again receives the fuel and oxygen needed to make energy. The intent of administering pyruvate during reperfusion is to stimulate the heart's contractile performance, that is, to help the heart beat stronger and pump more blood. The aim of this invention is to protect the heart during cardiac arrest, not to stimulate its function during that period which would interfere with surgery.

It should also be noted that the pyruvate-fortified cardioplegia is quickly washed out of the organ upon restoration of blood flow, so the improved function of the heart during the 12 hours following surgery is not due to a direct effect of pyruvate. Instead, the improved function is the result of excellent protection of the heart by pyruvate during the preceding ischemic period. Thus this mechanism is fundamentally different from pyruvate enhancement of cardiac performance in reperfusion.

We claim:

1. A cardioplegia solution comprising water with the following chemicals dissolved therein in the following concentrations:

| | |
|---|---|
| Pyruvate (mM) | 0.2-50 |
| NaCl (mM) | 0-250 |
| KCl (mM) | 0-250 |
| Glucose (mM) | 0-200 |
| Insulin (U/l) | 0-200 |
| CaCl₂ (mM) | 0-20 |
| Lidocaine (g/l) | 0-2. |

2. The solution of claim 1, in which pyruvate is provided in the chemical form of a free acid, as a salt in which the metal cation is sodium, calcium, or potassium, or as a salt in which the cation is an organic compound.

3. The solution of claim 1, additionally comprising amino acids.

4. The solution of claim 1, additionally comprising vitamins.

5. The solution of claim 1, additionally comprising a pharmacological agent.

6. The solution of claim 5, wherein the pharmacological agent is selected from the group consisting of β-adrenergic receptor antagonists, Ca²⁺ channel antagonists, and antioxidants.

7. A process for performing cardiopulmonary bypass surgery in a human comprising the following steps:
i) preparing a patient to allow surgical access to the heart;
ii) inducing arrest of the heart through application of a cardioplegia comprising the following chemicals dissolved therein in the following concentrations:

| | |
|---|---|
| Pyruvate (mM) | 0.2-50 |
| NaCl (mM) | 0-250 |
| KCl (mM) | 0-250 |
| Glucose (mM) | 0-200 |
| Insulin (U/l) | 0-200 |
| CaCl₂ (mM) | 0-20 |
| Lidocaine (g/l) | 0-2 |

iii) providing a bypass mechanism to a partially or wholly obstructed artery;
iv) ceasing administration of the cardioplegia solution;
v) providing a chemical or mechanical stimulus to induce the heart to resume beating;
vi) completing the surgical procedure.

8. The process of claim 7, wherein the cardioplegia solution is diluted with whole blood prior to administration to the patient.

9. The process of claim 8, wherein the ratio of blood volume to cardioplegia solution volume is between 0.1:1 and 20:1.

10. The process of claim 8, wherein the ratio of blood volume to cardioplegia solution volume is between 1:1 and 10:1.

11. The process of claim 8, wherein the ratio of blood volume to cardioplegia solution volume is between 2:1 and 8:1.

12. The process of claim 7, wherein the card ioplegia solution protects the heart from injury resulting from ischemia.

13. The process of claim 7, wherein the heart exhibits rapid and robust recovery of mechanical function following the provision of a mechanical or chemical stimulus to resume beating.

14. The process of claim 7, wherein the cardioplegia solution stabilizes the heart's energy reserves during the cardiopulmonary bypass surgery.

15. The process of claim 7, wherein metabolism of the cardioplegia solution by the heart produces compounds which neutralize prooxidant compounds during and immediately after the period of arrest.

16. The process of claim 7, wherein metabolism of the cardioplegia solution by the heart maintains the heart's antioxidant components during cardiopulmonary bypass surgery.

17. The process of claim 7, wherein pharmacological inotropic support is not administered following completion of the surgical procedure.

18. A method of preparing a cardioplegia solution comprising the steps of:
i) obtaining medical-grade electrolyte reagents and preparing in water a solution of the following reagents dissolved therein in the following concentrations:

| | |
|---|---|
| Pyruvate (mM) | 0.2-50 |
| NaCl (mM) | 0-250 |
| KCl (mM) | 0-250 |
| Glucose (mM) | 0-200 |
| Insulin (U/l) | 0-200 |
| CaCl₂ (mM) | 0-20 |
| Lidocaine (g/l) | 0-2; |

ii) filtering the solution through a filter with a pore size between of between 0.05 and 1 μm.
